(19) United States
(12) Patent Application Publication
(10) Pub. No.: US 2020/0046617 A1
(43) Pub. Date: Feb. 13, 2020

(54) Composition For Combating The Signs Of Ageing Of The Skin And Hair And Nails

(71) Applicants: TOMCAT INTERNATIONAL LIMITED, London (GB); PRESIDENT AND FELLOWS OF HARVARD COLLEGE, Cambridge, MA (US)

(72) Inventors: Mathilde Thomas, Paris (FR); Bertrand Thomas, Paris (FR); David Sinclair, Chestnut Hill, MA (US)

(21) Appl. No.: 16/608,063

(22) PCT Filed: Apr. 24, 2017
(86) PCT No.: PCT/IB2017/000703

§ 371 Date: Oct. 24, 2019

(51) Int. Cl. A61K 8/34; A61K 8/44; A61Q 19/08; A61Q 19/02

(52) U.S. Cl. CPC A61K 8/347; A61Q 19/02; A61Q 19/08; A61K 8/44

(57) ABSTRACT

The invention relates to a cosmetic use of an association of (i) resveratrol or a derivative thereof and (ii) betaine or a derivative thereof, or of a composition containing said association in a physiologically acceptable medium, to fight against the signs of ageing of the skin.

### FIELD OF THE INVENTION

This invention relates to a cosmetic treatment method for fighting against the signs of ageing of the skin and skin appendages, and improving the appearance of the skin. It also relates to compositions containing a synergic association to maintain the energy of the skin, and the synthesis of its main constituents, as well as the use of said association.

### REFERENCE TO SEQUENCE LISTING

This application contains a sequence listing submitted as an ASCII text file via EFS-Web having the file name 3225406-Sequence listing_ST25.TXT, created on Oct. 24, 2019 with a file size of 1,022 bytes. The contents of the Sequence Listing are incorporated herein by reference. This file does not go beyond the disclosure filed with International priority Patent Application No. PCT/IB2017/000703 filed Apr. 24, 2017.

### BACKGROUND OF THE INVENTION

The skin is composed of two layers, an outer layer called the epidermis and an inner layer called the dermis, which interact. Human epidermis is mainly composed of three types of cell: a large majority of keratinocytes, as well as melanocytes and Langerhans cells. Each of these cell types contributes through its specific functions to the role of the skin in the organism, and especially to protection against external attacks.

The epidermis is conventionally divided into a basal layer of keratinocytes forming the germinative layer of the epidermis, a spinous layer composed of several layers of polyhedral cells arranged on the germinative layers, one to three granular layers composed of flattened cells containing distinct cytoplasmic inclusions, the keratohyalin grains, and lastly the cornified layer (stratum corneum), composed of a set of keratinocyte layers at the final step of differentiation called corneocytes.

The dermis provides the epidermis with a solid support. It is also its nurturing element. It consists mainly of fibroblasts and an extracellular matrix composed mostly of collagen, elastin and a “fundamental” substance. These components are synthesised by the fibroblasts. There are also leucocytes, mast cells and tissular macrophages. Lastly, the dermis is traversed by blood vessels and nerve fibres.

The cohesion between the epidermis and the dermis is provided by the dermoepidermal junction.

Mitochondria are organelles present in most cells, where they play an essential role in the energy processes and cell respiration. With age, mitochondrial dysfunction contributes to the appearance of signs of ageing, especially of the skin.

The number or operation of the mitochondria may decrease with age.

In addition, it is known that the collagen and elastin present in the skin may also decrease over time in an individual, leading to the appearance of cutaneous signs such as wrinkles, fine lines and loss of skin elasticity.

Ageing phenomena, whether intrinsic or extrinsic, are natural normal phenomena, occurring in healthy individuals. Many men and women, however, feel the need to improve the appearance of their skin or their skin appendages and reduce or delay the signs related to ageing of the skin.

Resveratrol and derivatives thereof are antioxidant agents useful in cosmetics, giving good results in the fight against signs of cutaneous ageing.

Resveratrol derivatives have been described in patent application WO 99/03816, for therapeutic, cosmetic or dietary applications.

Patent application FR 3 017 289 describes compositions with high direct antioxidant power containing polyphenol and vitamin C derivatives.

Nevertheless, there is still a need to find new active ingredients, or associations of active ingredients, that are efficient in the fight against the signs of ageing of the skin or skin appendages.

Betaine, or tri-methylglycine, is used traditionally in cosmetics as a formulation excipient, surfactant or antistatic agent. It has also been proposed as moisturiser or as capillary conditioner.

FR2877843 relates to the use of betaine to fight against hydric or thermal stress.

WO 91/18588 describes the use of betaine to reduce skin irritation induced by cosmetic compositions. WO 2010/043769 also relates to the use of betaine to protect the skin from irritation.

DE102011089558 proposes the use of a mixture of betaine and urea derivatives to reinforce the skin barrier function.

However, to the applicant's knowledge, an effect of betaine to fight against the signs of ageing has never been described.

Surprisingly, it has now been demonstrated that betaine significantly increases the mitochondrial mass in cutaneous cells, in particular the cutaneous cells of elderly subjects.

### SUMMARY OF THE INVENTION

Unexpectedly, in the context of this invention it has been found that betaine may fight against some signs of ageing, by acting on various effects of age on the skin. All these effects are also obtained with resveratrol, which acts synergically with betaine.

This explains why it is an object of this invention to provide a cosmetic treatment method for fighting against the signs of ageing of the skin or skin appendages comprising a step of applying, on the cutaneous coating or its annexes, an association (i) of resveratrol or a derivative thereof and (ii) of betaine or a derivative thereof, or a composition containing them in a cosmetically acceptable medium.

The invention also relates to the use of an association (i) of resveratrol or a derivative thereof and (ii) of betaine or a derivative thereof, as agent to fight against the signs of ageing of the skin or skin appendages, especially by topical administration.

The invention therefore relates to the cosmetic use of an association (i) of resveratrol or a derivative thereof and (ii) of betaine or a derivative thereof, or of a composition containing said association in a physiologically acceptable medium, to fight against the signs of ageing of the skin.

The uses and methods according to the invention are, unless stipulated otherwise, cosmetic and non-therapeutic; their purpose is essentially aesthetic.

The invention further relates to a composition, useful for the implementation of the cosmetic treatment method, containing an association (i) of resveratrol or a derivative thereof and (ii) of betaine, in a physiologically acceptable medium, in particular a cosmetically acceptable medium.

In particular, one aspect of the invention is the cosmetic use of an association of (i) resveratrol or a derivative thereof and (ii) betaine or a derivative thereof, or of a composition containing said association in a physiologically acceptable medium, to fight against the signs of ageing of the skin.

In some embodiments, the association or composition containing it is administered orally.

In certain embodiments, the association or composition containing it is applied topically on the skin.

In some embodiments, the association (i) of resveratrol or a derivative thereof and (ii) of betaine or a derivative thereof, or of the composition is used to delay and/or reduce and/or prevent the signs of ageing selected from loss of skin firmness, thinning of the skin, wrinkles, fine lines, dull complexion and pigmentary spots.

In certain embodiments, the use is to stimulate the synthesis of collagen and/or elastin in the skin.

In some advantageous embodiments, the association (i) of resveratrol or a derivative thereof and (ii) of betaine or a derivative thereof has a synergic effect on the energy metabolism and/or the production of energy by the skin cells.

In preferred embodiments, resveratrol or its derivatives (i) is selected from cis-resveratrol, trans-resveratrol and the resveratrol esters, monomers and oligomers, the monomers comprising at least one ester group of formula —O—CO-A, and the oligomers being formed from monomer units linked by carbon-carbon or ether bonds, and/or of monomers cross-linked with —O—CO—R—CO—O— groups where -A represents a linear or branched, saturated or unsaturated alkyl radical with at least two carbon atoms, an aryl, aralkyl or aralkylene radical, and —R represents a saturated or unsaturated alkylene radical having 0 to 10 carbon atoms and/or 1 arylene radical having 1 to 3 rings and/or a heterocyclic radical and the diastereoisomers of these units.

In some embodiments, the association is in the form of a conjugate of betaine with resveratrol or a derivative thereof.

In another aspect, the invention provides a cosmetic treatment method for fighting against the signs of ageing of the skin comprising a step of applying, on the cutaneous coating or its annexes, a composition containing an association (i) of resveratrol or a derivative thereof and (ii) of betaine or a derivative thereof in a cosmetically acceptable medium.

In some embodiments the compositions or methods can be used to delay and/or reduce and/or prevent the signs of ageing selected from loss of skin firmness and/or elasticity, thinning of the skin, wrinkles and fine lines, dull complexion, skin hyperpigmentation or pigmentation heterogeneity and pigmentary spots.

In certain embodiments, resveratrol or derivatives thereof are present in the composition in the form of a plant extract containing it.

In some embodiments, betaine is in the form of one of its physiologically acceptable salts.

In certain embodiments, the ratio between resveratrol and betaine is between 5:1 and 1:1000.

In some embodiments, the inventive composition contains (i) from 0.001 to 10% of resveratrol and (ii) from 0.01 to 10% of betaine, the percentages being expressed in weight relative to the total weight of the composition.

In yet another aspect, the invention provides a composition for the aforementioned uses, or useful for the aforementioned methods, characterised in that it contains, in a physiologically acceptable medium, a synergic association on the production of collagen by the cutaneous cells of (i) resveratrol or a derivative thereof and of (ii) betaine, resveratrol or its derivatives being present at a concentration of 0.002 to 3% and betaine being present at a concentration of 0.1 to 10%, the percentages being expressed in weight relative to the total weight of the composition.

In some embodiments, the medium is a cosmetically acceptable medium and in that it further contains at least one active ingredient selected from the antioxidant agents, hydrating agents, exfoliating agents, agents improving the barrier function, depigmenting agents and agents promoting cutaneous microcirculation.

The embodiments of the present invention will be described with reference to the accompanying drawings and below description.

### BRIEF DESCRIPTION OF THE DRAWINGS

FIG. 1 is a chart showing the effect of resveratrol and/or betaine, alone or in combination, on ATP production by cultivated human fibroblasts; the association of resveratrol and betaine has a synergic effect on the increase of ATP production.

### DETAILED DESCRIPTION OF THE INVENTION

The invention is directed in particular to compositions wherein resveratrol or derivatives thereof have the following formula:

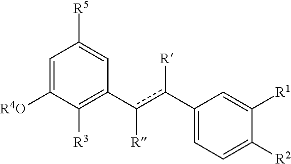

### Wherein

R′ is a hydrogen or the junction point at R3 or at R4 of another unit,

R″ is a hydrogen or the junction point at R3 or at R″ of another unit,

R1 is a hydrogen, an alcohol group, an ether group or an ester group,

R2 is a hydrogen, an alcohol group, an ether group or an ester group,

R3 represents a hydrogen or the junction point at R″ or at R′ of another unit,

R4 is a hydrogen, an alkyl radical, an acyl radical or the junction point at R′ of another unit

R5 is a hydrogen, an alcohol group, an ether group or an ester group, and the diastereoisomers and regioisomers of these moieties.

Trans-resveratrol, trans-rhapontin, trans-desoxyrhapontin, trans-piceatannol, trans-astringin, trans-pterostilbene, trans-pinosylvine, trans-piceide, trans-ε-viniferin, and trans-miyabenol C are examples of such compounds.

For the purposes of this invention, “elderly subjects” refers to human individuals over 30 years old, in particular over 40 years old, and advantageously over 50 years old, or even over 60 years old.

It is known that ageing in mammals and more especially in human results in mitochondrial alterations. In addition, the number of mitochondria in the cells decreases with age.

Mitochondria produce energy in the cells by contributing to ATP production via β-oxidation, the Krebs cycle and the respiratory chain during oxidative phosphorylation. Ageing is accompanied by a reduction in the activity of the respiratory chain, resulting in a reduction of ATP production. This reduction has been estimated at 8% per decade.

ATP is also essential in numerous processes required to ensure correct cellular operation, and in particular to maintain protein synthesis activities, including the synthesis of proteins of the extracellular matrix.

Mitochondrial dysfunctions and in particular the reduction of respiratory activity in the skin cells may therefore favour a loss of skin softness, flexibility and/or elasticity, the formation of wrinkles, fine lines, dull and/or non-homogeneous complexion, and a loss of skin radiance or transparency.

Betaine can also stimulate the metabolism of the mitochondria and more especially restore the energy of the cell.

This activity is obtained synergically for an association of betaine and resveratrol, or of its derivatives.

Synergy means that the effect obtained with the association of the two active ingredients is greater than the sum of the effects obtained with each active ingredient taken individually.

The association (i) of betaine or derivatives thereof and (ii) of resveratrol or derivatives thereof has a synergic effect on the restoration and/or preservation of the skin energy metabolism. The association has a synergic effect on the stimulation of the energy metabolism and/or the production of energy by the skin cells.

An association according to the invention, or a composition containing it, is therefore useful to stimulate the synthesis of collagen and/or elastin by the skin cells.

Thus, in the context of this invention, it has been demonstrated that the synthesis of collagen and elastin by the skin cells, such as the fibroblasts, is stimulated firstly by resveratrol.

Betaine also has a stimulating effect on the synthesis of collagen by the cutaneous cells. The association of betaine and resveratrol or derivatives thereof has a synergic effect on this stimulation.

The invention therefore relates to the use of the association (or the compositions containing it), to fight against the signs of ageing of the skin or skin appendages, whether intrinsic or extrinsic.

Apart from wrinkles and fine lines, the appearance of pigmentary spots, reduction in skin thickness, loss of elasticity and sagging are also changes observed during ageing.

The association, or a composition containing it, will be used in particular to reduce or prevent cutaneous disorders such as loss of skin firmness and/or elasticity, thinning of the skin, wrinkles and fine lines, dull complexion, skin hyperpigmentation or pigmentation heterogeneity and pigmentary spots.

Skin means the skin of the face and/or of the body, the scalp and the mucous or semi-mucous membranes (such as the lips). Skin appendages means the hair, hairs, eyelashes, nails, and preferably the hair.

The method according to the invention can be used in particular for delaying and/or reducing and/or preventing the signs of ageing selected from loss of skin firmness, thinning of the skin, wrinkles, fine lines, dull complexion and pigmentary spots.

In this description, for intervals expressed “between . . . and . . . ” or “from . . . to . . . ”, the limits are understood to be included.

Resveratrol or derivatives thereof adapted to implementation of the invention are selected in particular from cis-resveratrol, trans-resveratrol, viniferins, and the resveratrol esters, monomers and oligomers, the monomers comprising at least one ester group of formula —O—CO-A, and the oligomers being formed from monomer units linked by carbon-carbon or ether bonds, and/or of monomers cross-linked with —O—CO—R—CO—O— groups

where -A represents a linear or branched, saturated or unsaturated alkyl radical with at least two carbon atoms, an aryl, aralkyl or aralkylene radical, and

—R represents a saturated or unsaturated alkylene radical having 0 to 10 carbon atoms and/or 1 arylene radical having 1 to 3 rings and/or a heterocyclic radical and the diastereoisomers of these units.

Such derivatives are described in application WO 9903816.

In one embodiment of the invention, the compositions are based on resveratrol monomers and/or oligomers comprising at least one —O—CO-A group. In a preferred group, A represents a saturated or unsaturated fatty acid radical.

In case of unsaturation, the double bonds are advantageously cis, which corresponds to the most frequent case in natural products. With products obtained more particularly by synthesis or hemisynthesis, the bonds are trans.

The fatty acids suitable for implementation of the invention include the butyric, valeric, hexanoic, sorbic, lauric, palmitic, stearic, oleic, linoleic, linolenic, α-linolenic, arachidonic, eicosapentaenoic and docosahexaenoic acids.

Fatty acids with 16 or more carbon atoms are especially suitable for cosmetic applications.

In another preferred group, A represents an aryl group, such as the phenyl radical.

In yet another group, A represents an aralkyl or aralkylene group, the alkyl or alkylene group being more particularly from C1 to C8, especially C1 to C4. We may mention, in particular, the benzyl or styryl group.

Resveratrol, or 3,5,4′-trihydroxystilbene, exists in both cis and trans forms. It generally takes the monomer form but also exists as an oligomer with up to four monomers. Unless otherwise indicated, resveratrol refers to the cis or trans forms of 3,5,4′-trihydroxystilbene, and mixtures thereof. Resveratrol is often associated with ε-viniferin which is a dimer of resveratrol.

Advantageously, the trans form of resveratrol will be used. Other derivatives which can be used include rhapontin, deoxyrhapontin, piceatannol, astringin, pterostilbene, pinosylvin, piceid, and viniferins.

Oligomers especially suitable for the invention include for example from 2 to 4 monomer units.

According to a special embodiment, all the hydroxyl groups of resveratrol are esterified or etherified, so the derivative therefore no longer has any free OH function.

Such ethers or esters may in particular be prepared using the method described in patent applications WO 2011/128714 and WO 99/03816.

According to an embodiment of the invention, resveratrol or derivatives thereof are used in the form of plant extracts containing it.

They can be obtained from various plant sources. We may mention Vitaceae, Umbelliferae, Myrtaceae, Dipterocarpaceae, Cyperaceae, Gnetaceae, Leguminosae, Gramineae, Sericeae, Haemodoraceae, Musaceae, Polygonaceae, Pinaceae, Cupressaceae, Cesalpiniaceae, Poaceae, and Solanaceae.

In particular, resveratrol and or the resveratrol derivatives used in the associations of the invention are vine extracts, and more especially grape shoot extracts; these extracts contain monomers, oligomers and/or polymers of units having the above formula.

Advantageously, the monomers and/or oligomers of resveratrol used are as obtained by extraction, with water and/or an organic solvent, from vine stalks.

Resveratrol can also be obtained through the use of genetically modified microorganisms, from animal tissue or by bioproduction from plants, in particular from suspensions of vine cells.

Betaine, or trimethylglycine, can be used in the form of zwitterion, or in the form of physiologically acceptable salts or derivatives thereof.

In the present text, betaine or derivatives thereof should be understood as designating betaine, in the form of zwitterion or in the form of physiologically acceptable salts thereof (for instance betaine HCl) and/or in the form of physiologically acceptable derivatives of betaine; betaine can be present in the compositions according to the invention in any of these forms, and/or their mixtures in all proportions.

The association useful for the implementation of the invention may contain resveratrol in the form of its various isomers or one of its derivatives as defined above, individually or mixed in all proportions.

According to one embodiment of the invention, the association of betaine and resveratrol or derivatives thereof is in the form of a conjugate of betaine molecule with resveratrol or derivatives thereof.

In particular, resveratrol or derivative thereof may be covalently linked with betaine, for instance via an ester linkage.

The association all along the present specification, encompass said conjugate, resulting from the conjugation between betaine and resveratrol or its derivatives.

The association of (i) resveratrol or a derivative thereof and of (ii) betaine has a synergic effect on increasing the mitochondrial activity, and in particular, the mitochondrial mass, in the skin cells, and more specifically in the cutaneous fibroblasts, especially in fibroblasts under condition mimicking that of fibroblasts of an elderly subject.

The mitochondrial mass is therefore restored to that of a young cell.

The association according to the invention also has a synergic effect on the Trans-resveratrol, trans-rhapontin, trans-desoxyrhapontin, trans-piceatannol, trans-astringin, trans-pterostilbene, trans-pinosylvine, trans-piceide, trans-ε-viniferin, and trans-miyabenol C are examples of such compounds.

In one embodiment of the invention, the compositions are based on resveratrol monomers and/or oligomers comprising at least one —O—CO-A group. In a preferred group, A represents a saturated or unsaturated fatty acid radical.

In case of unsaturation, the double bonds are advantageously cis, which corresponds to the most frequent case in natural products. With products obtained more particularly by synthesis or hemisynthesis, the bonds are trans.

The fatty acids suitable for implementation of the invention include the butyric, valeric, hexanoic, sorbic, lauric, palmitic, stearic, oleic, linoleic, linolenic, α-linolenic, arachidonic, eicosapentaenoic and docosahexaenoic acids.

Fatty acids with 16 or more carbon atoms are especially suitable for cosmetic applications.

In another preferred group, A represents an aryl group, such as the phenyl radical.

In yet another group, A represents an aralkyl or aralkylene group, the alkyl or alkylene group being more particularly from C1 to C8, especially C1 to C4. We may mention, in particular, the benzyl or styryl group.

Resveratrol, or 3,5,4′-trihydroxystilbene, exists in both cis and trans forms. It generally takes the monomer form but also exists as an oligomer with up to four monomers. Unless otherwise indicated, resveratrol refers to the cis or trans forms of 3,5,4′-trihydroxystilbene, and mixtures thereof. Resveratrol is often associated with ε-viniferin which is a dimer of resveratrol.

Advantageously, the trans form of resveratrol will be used. Other derivatives which can be used include rhapontin, deoxyrhapontin, piceatannol, astringin, pterostilbene, pinosylvin, piceid, and viniferins.

Oligomers especially suitable for the invention include for example from 2 to 4 monomer units.

According to a special embodiment, all the hydroxyl groups of resveratrol are esterified or etherified, so the derivative therefore no longer has any free OH function.

Such ethers or esters may in particular be prepared using the method described in patent applications WO 2011/128714 and WO 99/03816.

According to an embodiment of the invention, resveratrol or derivatives thereof are used in the form of plant extracts containing it.

They can be obtained from various plant sources. We may mention Vitaceae, Umbelliferae, Myrtaceae, Dipterocarpaceae, Cyperaceae, Gnetaceae, Leguminosae, Gramineae, Sericeae, Haemodoraceae, Musaceae, Polygonaceae, Pinaceae, Cupressaceae, Cesalpiniaceae, Poaceae, and Solanaceae.

In particular, resveratrol and or the resveratrol derivatives used in the associations of the invention are vine extracts, and more especially grape shoot extracts; these extracts contain monomers, oligomers and/or polymers of units having the above formula.

Advantageously, the monomers and/or oligomers of resveratrol used are as obtained by extraction, with water and/or an organic solvent, from vine stalks.

Resveratrol can also be obtained through the use of genetically modified microorganisms, from animal tissue or by bioproduction from plants, in particular from suspensions of vine cells.

Betaine, or trimethylglycine, can be used in the form of zwitterion, or in the form of physiologically acceptable salts or derivatives thereof.

In the present text, betaine or derivatives thereof should be understood as designating betaine, in the form of zwitterion or in the form of physiologically acceptable salts thereof (for instance betaine HCl) and/or in the form of physiologically acceptable derivatives of betaine; betaine can be present in the compositions according to the invention in any of these forms, and/or their mixtures in all proportions.

The association useful for the implementation of the invention may contain resveratrol in the form of its various isomers or one of its derivatives as defined above, individually or mixed in all proportions.

According to one embodiment of the invention, the association of betaine and resveratrol or derivatives thereof is in the form of a conjugate of betaine molecule with resveratrol or derivatives thereof.

In particular, resveratrol or derivative thereof may be covalently linked with betaine, for instance via an ester linkage.

The association all along the present specification, encompass said conjugate, resulting from the conjugation between betaine and resveratrol or its derivatives.

The association of (i) resveratrol or a derivative thereof and of (ii) betaine has a synergic effect on increasing the mitochondrial activity, and in particular, the mitochondrial mass, in the skin cells, and more specifically in the cutaneous fibroblasts, especially in fibroblasts under condition mimicking that of fibroblasts of an elderly subject.

The mitochondrial mass is therefore restored to that of a young cell.

The association according to the invention also has a synergic effect on the production of energy by the cells of the skin or its annexes, in particular by fibroblasts of human skin.

This effect is observed in particular on ATP production by these cells.

It is known that ATP production is a major source of energy for the various cell processes and that its production decreases with age.

Resveratrol (or a derivative thereof), on one hand, and betaine, on the second hand, can be used to increase ATP production by fibroblasts. The association according to the invention has a synergic effect on the increase of ATP production.

In particular, on fibroblasts of human skin, intracellular ATP synthesis is increased by at least 10%, in particular by at least 18%, even by at least 20%, compared with the increase which would be expected by simply adding the individual effects of each of the two components of the association.

The ATP level can be restored to that of young cells in a subject in good health.

The skin cells may therefore recover the metabolism of young cells, and in particular protein synthesis is restored.

According to one aspect of the invention, resveratrol, or derivatives thereof, increase the production of collagen and/or elastin by the cutaneous cells, in particular by fibroblasts of human skin.

Betaine also increases the production of collagen by the cutaneous cells, in particular by fibroblasts of human skin.

The association according to the invention, or the composition containing it, will therefore be useful to stimulate the synthesis of collagen and/or elastin in the skin.

Advantageously, the association according to the invention has a synergic effect on increasing the production of collagen by fibroblasts of the skin, in particular human skin.

The collagen production is therefore equivalent to that of a young skin. For example, the collagen content in a subject to which the association or the composition containing it is administered, is restored to the average collagen content in a younger subject.

According to one embodiment of the invention, the ratio between resveratrol and betaine is between 5:1 and 1:1000, for instance 5:1 and 1:500 and in particular between 1:1 and 1:500.

The compositions useful for the implementation of the invention contain for example (i) from 0.001 to 10% of resveratrol, in particular from 0.001 to 3% of resveratrol and for instance 0.002 to 1% and (ii) from 0.01 to 10% of betaine, in particular from 0.01 to 5% and for instance from 0.1 to 5%, the percentages being expressed in weight relative to the total weight of the composition.

In particular, in a composition according to the invention, resveratrol and/or derivatives thereof can be present in a concentration from 0.002 to 3%, for instance from 0.002 to 0.5%, in weight relative to the total weight of the composition.

According to one embodiment of the invention, the association (i) of resveratrol or derivatives thereof and (ii) of betaine or the composition containing it, is administered orally.

According to another embodiment, the association (i) of resveratrol or derivatives thereof and (ii) of betaine or the composition containing it, is applied topically on the skin.

These two embodiments can be combined, the association and/or the composition containing it can be administered orally and topically simultaneously, separately, or sequenced over time.

A composition especially suited to the invention contains, in a physiologically and in particular cosmetically acceptable medium, a synergic association on the production of collagen by the cutaneous cells of (i) resveratrol or a derivative thereof and of (ii) betaine in such composition, resveratrol or its derivatives may be present at a concentration of 0.01 to 3% and betaine may be present at a concentration of 0.1 to 10%, the percentages being expressed in weight relative to the total weight of the composition.

The compositions according to the invention are preferably cosmetic compositions, or dermatological compositions adapted to an application on the skin or skin appendages. The composition according to the invention contains the association of resveratrol or derivatives thereof and betaine as defined previously, and a physiologically acceptable medium.

Physiologically acceptable medium means a medium compatible with the skin or skin appendages and not causing any undesirable effects when placed in contact with the various parts of the body. It can be a medium compatible with oral administration.

It is in particular a cosmetically acceptable medium. This therefore means a medium or excipients well tolerated by the skin and having a pleasant smell or appearance.

For oral administration, the composition of the invention may take any suitable form, in particular the form of a drinkable solution, a syrup, tablet, softgel, dragee, capsule, controlled release hydrogel, or a nutritional food or a nutritional supplement. In particular, the association can be incorporated in any form of nutritional supplement, for example food bars or powders compacted or not.

For oral use, the composition may comprise an ingestible carrier.

Cosmetic product or composition means a substance or mixture intended to be placed in contact with the external parts of the human body or with the teeth and the mucous membranes of the oral cavity, with a view exclusively or mainly to cleaning them, perfuming them, changing their appearance, protecting them, keeping them in good condition or correcting body odours. A cosmetic treatment method according to the invention has the same objectives.

This composition may be more or less fluid and have the appearance of a white or coloured cream, an ointment, a milk, a lotion, a serum, a paste, or a foam. It may also take the form of a solid, in particular as a stick. It can be used as a care product and/or as a make-up product and/or a hygiene product for the skin or skin appendages.

The composition according to the invention may take any galenic form normally used in the field of cosmetics, and it may take in particular the form of an aqueous or oily solution, possibly gelled, a dispersion of the lotion type, possibly two-phase, an emulsion obtained by dispersion of an oil phase in an aqueous phase (O/W) or inversely (W/O), or a triple emulsion (W/O/W or O/W/O) or a vesicular dispersion of ionic or non-ionic type. These compositions are prepared according to the usual methods.

When the composition used according to the invention is an emulsion, the proportion of the oil phase may range from 0.5 to 80% by weight, and preferably from 5 to 50% by weight relative to the total weight of the composition. The oils, emulsifiers and co-emulsifiers used in the composition as an emulsion are selected from those traditionally used in the field considered. The emulsifier and co-emulsifier are present, in the composition, in a proportion ranging from 0.1 to 30% by weight, and preferably from 0.5 to 20% by weight relative to the total weight of the composition.

The oils which can be used in the invention include mineral (mineral oil) or synthetic (vaseline oil, isohexadecane) hydrocarbons, vegetable oils (apricot seed oil, grape seed oil, shea butter liquid fraction, avocado oil, soybean oil), animal oils (lanolin), synthetic oils (perhydrosqualene, pentaerythrityl tetraisononanoate), silicon-containing derivatives (cyclopentasiloxane, cyclohexasiloxane, dimethicone and silicon polymers) and fluorinated oils (perfluoropolyethers). Fatty alcohols (cetyl or stearyl alcohol), fatty acids (stearic acid), waxes (carnauba wax, ozokerite, beeswax), butters and hydrogenated oils can also be used as fats.

The emulsifiers and co-emulsifiers which can be used in the invention include, for example, the fatty acid and polyethylene glycol esters such as PEG-100 stearate and PEG-20 stearate and the fatty acid and glycerin esters such as glyceryl stearate, sucrose esters and phospholipids.

In a known manner, the composition used according to the invention may also contain the additives generally found in the field of cosmetics, such as hydrophilic or lipophilic gelling agents, hydrophilic or lipophilic ingredients, preservatives, antioxidants, solvents, perfumes, fillers, filters, screens, pigments, odour absorbers, chelating agents, alcohols and colorants. The quantities of these various additives are those traditionally used in the field considered, and for example from 0.01 to 20% of the total weight of the composition. These additives, depending on their type, may be introduced in the oil phase, the aqueous phase or in the lipid vesicles. These additives and their proportions will always be selected so as not to impair the desired properties of the association.

The hydrophilic gelling agents include in particular the carboxyvinyl polymers (carbomer), acrylic copolymers such as acrylates/alkylacrylates copolymers, polyacrylamides, polysaccharides, natural gums and clays, and the lipophilic gelling agents include the modified clays such as bentones, fatty acid metal salts, hydrophobic silica and polyethylenes.

The fillers include, for example, polyamide (Nylon) particles either spherical or as microfibres; methyl polymethacrylate microspheres; ethylene-acrylate copolymer powders; expanded powders such as hollow microspheres and in particular microspheres formed from a terpolymer of vinylidene chloride, acrylonitrile and methacrylate and sold under the trade name EXPANCEL by the company Kemanord Plast; powders of natural organic materials such as starch powders, in particular maize, wheat or rice starches, cross-linked or not, such as starch powders cross-linked with octenyl succinate anhydride; silicone resin microbeads such as those sold under the name TOSPEARL by the company Toshiba Silicone; silica; metal oxides such as titanium dioxide or zinc oxide; mica; and mixtures thereof.

The composition used according to the invention may further contain other active ingredients and in particular at least one compound selected from: antioxidant agents, hydrating agents; exfoliating agents; agents improving the barrier function; agents promoting cutaneous microcirculation; depigmenting agents; anti-glycation agents; NO-synthase inhibitors; agents stimulating the synthesis of dermal or epidermal macromolecules and/or preventing their degradation; agents stimulating the proliferation of fibroblasts and/or keratinocytes or stimulating the differentiation of keratinocytes; myorelaxants; tensor agents; anti-pollution and/or anti-radical agents, solar filters, propigmenting agents, anti-inflammatory agents, agents modifying sebum production, refreshing agents and mixtures thereof.

The antioxidant agents include in particular tocopherol and esters thereof, especially tocopherol acetate; ascorbic acid and derivatives thereof, especially magnesium ascorbyl phosphate, ascorbyl glucoside and ascorbyl tetraisopalmitate; ferulic acid; serine; ellagic acid, phloretin, chelating agents, such as BHT, BHA, N,N′-bis(3,4,5-trimethoxybenzyl) ethylenediamine and salts thereof, hydroxymataisoresinol and mixtures thereof.

In particular, the cosmetic composition according to the invention further contains at least one active ingredient selected from vitamin C and derivatives thereof and vitamin E or derivatives thereof such as tocopherol acetate.

The additional active ingredients may be present in the composition according to the invention in a content ranging from 0.001% to 20% by weight relative to the total weight of the composition, preferably from 0.01% to 10%, even more preferably from 0.5% to 5% and even more preferably from 0.1% to 1% by weight relative to the total weight of the composition.

The invention also relates to a cosmetic treatment method comprising a step of applying on the skin or skin appendages the association or compositions according to the invention.

The objective of the cosmetic treatment method will therefore be to prevent or reduce the cutaneous signs such as loss of skin firmness and/or elasticity, thinning of the skin, wrinkles and fine lines, dull complexion, skin hyperpigmentation or pigmentation heterogeneity and pigmentary spots.

The compositions and associations according to the invention can be applied directly on the skin or skin appendages, or, alternatively, on occlusive or non-occlusive cosmetic or dermatological carriers, intended to be applied locally on the skin, such as hydrogel masks, wipes, fabrics, or biocellulose.

The application may for instance be carried out once a day, several times a day or once a week. It may continue for several days and/or several weeks, possibly longer; it may be continuous or for example continued for 1 to 2 months then resumed after an interruption.

The invention also relates to the use of an association of (i) resveratrol or a derivative thereof and (ii) of betaine or a derivative thereof, as defined herein, for the preparation of a cosmetic composition. The composition is adapted for an application on the skin or skin appendages. The composition can be used advantageously to fight against the signs related to the action of free radicals on the skin or skin appendages, restore the cell energy metabolism, stimulate the production of collagen or elastin by the skin and/or fight against the signs of skin ageing.

The invention also relates to a method for preparing a cosmetic composition comprising a step in which an efficient amount (i) of resveratrol or a derivative thereof and (ii) of betaine or a derivative thereof, and optionally a physiologically acceptable excipient are mixed.

Other features and advantages of the invention are illustrated in the following examples.

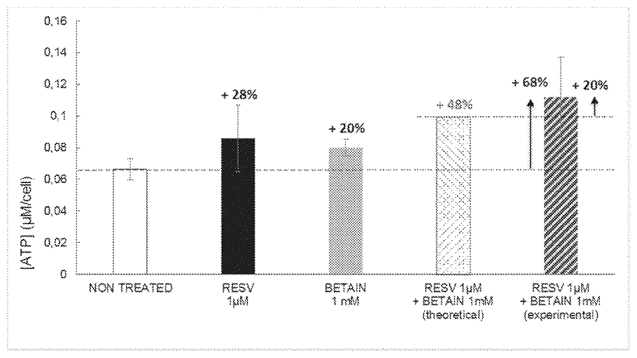

In these examples, it will be referred to the single FIGURE, FIG. 1.

FIG. 1 shows the effect of resveratrol and/or betaine, alone or in combination, on ATP production by cultivated human fibroblasts; the association of resveratrol and betaine has a synergic effect on the increase of ATP production.

### Example 1: Action of an Association of Betaine and Trans-Resveratrol on ATP Production

Human fibroblasts were cultured at 37° C. in wet atmosphere with 5% CO2, on MEM supplemented with glucose, serum and glutamine. A solution containing trans-resveratrol and/or betaine is added to the culture medium:

The primary fibroblast cultures are incubated for 120 min with resveratrol at a final concentration of 1 μM, betaine at a concentration of 1 mM or a mixture of the two.

The cell viability is measured after incubation by fluorescence. The intracellular ATP concentration is measured by a chemiluminescence-based assay; the mean of 4 measurements is taken.

The intracellular ATP concentrations are expressed in μM of ATP per viable cell and percent of ATP increase is calculated with respect to the ATP level in untreated cells.

The results are shown in the attached FIGURE and provided in the following table.

ATP (μM per cell)

| | Mean value | Increase (%) |
|---|---|---|
| Untreated | 0.067 | Reference |
| Resveratrol 1 μM | 0.086 | 28 |
| Betaine 1 mM | 0.080 | 20 |
| Resveratrol 1 μM + | 0.099 | 48 |
| Betaine 1 mM | | |
| (theoretical) | | |
| Resveratrol 1 μM + | 0.112 | 68 |
| Betaine 1 mM | | |
| (measured) | | |

The association of resveratrol and betaine significantly increases the intracellular ATP production, which is 68% greater than that of untreated cells.

The activity measured for the resveratrol+betaine association is more than 20% greater than the theoretical sum of the effects of each compound.

The association of betaine and resveratrol may combat the decrease of intracellular ATP concentrations in fibroblasts which is associated with ageing.

### Example 2: Increase in Mitochondrial Mass by Resveratrol and Betaine

Human fibroblasts were cultured at 37° C. in wet atmosphere with 5% CO2, on MEM supplemented with glucose, serum and glutamine. The cells are treated with a chemical agent (FK866) in the presence or absence of the following compounds: Trans-resveratrol and Betaine. Agent FK866 reduces the cell mitochondrial mass, thereby mimicking one of the signs of ageing.

Under these conditions, the ability of the active ingredient to increase the mitochondrial mass is assessed.

After 24 h treatment, the mitochondrial mass is therefore measured using the Nonyl Acridine Orange (NAO, Invitrogen) probe which fluoresces at 533 nm. The cell viability can be measured by DAPI staining. A flow cytometry analysis is conducted with a FACS LSRII and the results analysed by FlowJo software.

The results are provided in the following tables:

Treatment with FK866 decreases the mitochondrial mass of fibroblasts, mimicking effects of ageing

Mitochondrial mass
(fluorescence units)

| | Mean | Decrease (%) |
|---|---|---|
| Basal | 1468 | Reference |
| FK866 2.5 nM | 1326 | 10 |

Treatment with resveratrol, betaine and their association protects against the effects of FK866 on mitochondrial mass

Mitochondrial mass
(fluorescence units)

| | | Mean | Increase (%) |
|---|---|---|---|
| FK866 | Non treated | 1326 | Reference |
| 2.5 nM | Betaine 5 mM | 1574 | 19 |
| | Resveratrol | 2285 | 72 |
| | 25 μM | | |
| | Resveratrol | 2533 | 91 |
| | 25 μM + | | |
| | Betaine 5 mM | | |
| | (theoretical) | | |
| | Resveratrol | 2665 | 101 |
| | 25 μM + | | |
| | Betaine 5 mM | | |
| | (measured) | | |

### Example 3 Increase in Collagen Production by Resveratrol and Betaine

Cutaneous fibroblasts were cultured for 24 h with resveratrol, betaine or mixtures of the two compounds.

For the gene expression analysis, the total RNAs were extracted from the fibroblast cultures and the expression measured by quantitative real-time PCR. RNA extraction was carried out using an RNeasy Mini Kit (Qiagen). The total RNAs were used for the cDNA synthesis using the iScript cDNA synthesis Kit (Biorad). The PCR reactions were conducted using the mix LightCycler® 480 SYBR Green I Master (Roche). Each sample was subjected to two PCRs using the appropriate primers listed in the table below. To compare the relative expressions, the Δ ΔCt method was used. The relative expression of the gene of interest is represented by the expression variation compared with the untreated samples.

| Gene | Primer pairs |
|---|---|
| Col1A1 | GAG GGC CAA GAC |
| | GAA GAC ATC |
| | (SEQ ID NO: 1) |
| | CAG ATC ACG TCA |
| | TCG CAC AAC |
| | (SEQ ID NO: 2) |
| 18S RNA | GAG ACT CTG GCA |
| | TGC TAA CTA G |
| | (SEQ ID NO: 3) |
| | GGA CAT CTA AGG |
| | GCA TCA CAG |
| | (SEQ ID NO: 4) |

The results are provided in the following table:

Relative expression
of Col1A1

| | Mean value | Increase (%) |
|---|---|---|
| Untreated | 1.00 | Reference |
| Resveratrol 4 μM | 1.22 | 22 |
| Betaine 5 mM | 1.28 | 28 |
| Resveratrol 4 μM + | 1.50 | 50 |
| Betaine 5 mM | | |
| (theoretical) | | |
| Resveratrol 4 μM + | 1.54 | 54 |
| Betaine 5 mM | | |
| (measured) | | |

Resveratrol and betaine have a synergic effect on increasing the expression of collagen genes 1 and 3

### Example 4: Compositions

Composition according to the invention are prepared by mixing the components of the following formulations

Gel

| | % |
|---|---|
| Aqua | QSP 100 |
| Sodium phytate | 0.05 |
| Carbomer | 0.2 |
| Polyacrylate crosspolymer-6 | 0.2 |
| Caprylyl glycol | 0.3 |
| Palmitoyl grape shoot extract | 0.1 |
| Hydroxyethyl acrylate/sodium | 0.2 |
| acryloyldimethyltaurate copolymer, | |
| Aqua, Sorbitan isostearate | |
| Tocopherol, Sunflower seed oil | 0.3 |
| Betaine | 0.5 |
| Fragrance | 0.1 |
| | QSP pH 5.0-5.5 |

Cream Gel

| | % |
|---|---|
| Aqua | QSP 100 |
| Tetrasodium EDTA | 0.05 |
| Sodium acrylates copolymer, Lecithin | 2 |
| Sweet almond oil | 10 |
| Palmitoyl grape shoot extract | 0.35 |
| Potassium sorbate | 0.3 |
| BHT | 0.05 |
| Betaine | 0.5 |
| Fragrance | 0.3 |

Lotion

| | % |
|---|---|
| Aqua | QSP 100 |
| Glycerin | 10 |
| Resveratrol | 0.01 |
| Betaine | 1 |
| Alcohol | 15 |

Emulsion

| | % |
|---|---|
| Aqua | QSP 100 |
| Butylene glycol | 5 |
| Xanthan gum | 0.2 |
| Polyacrylate crosspolymer-6 | 0.5 |
| Cetearyl glucoside, cetearyl alcohol | 4 |
| Glyceryl stearate | 1 |
| Dicaprylyl ether | 5 |
| Butyrospermum parkii | 3 |
| Dimethicone | 4 |
| Carpyly/capric triglycerides | 10 |
| Silica | 2 |
| Chlorphenesin | 0.2 |
| Sodium benzoate | 0.2 |
| Palmitoyl grape shoot extract | 0.7 |
| Betaine | 0.3 |
| Tocopherol | 0.2 |
| Fragrance | 0.4 |

Throughout this application various publications, patents and/or patent applications have been referenced. The disclosure of these publications in their entireties is hereby incorporated by reference in this application.

The term comprising is intended herein to be open-ended, including not only the recited elements, but further encompassing any additional elements.

A number of embodiments have been described. Nevertheless, it will be understood that various modifications may be made. Accordingly, other embodiments are within the scope of the following claims.

1.-16. (canceled)

17. A method of treating aging skin comprising administering to a person in need thereof a composition including
(i) resveratrol or a derivative thereof,
(ii) betaine or a derivative thereof, and
(iii) a physiologically acceptable medium.

18. The method of claim 17 wherein, the composition containing is administered orally.

19. The method of claim 17, wherein the composition is administered by applying topically on the skin.

20. The method of claim 17, wherein the composition is administered to treat one or more of loss of skin firmness, thinning of the skin, wrinkles, fine lines, dull complexion and pigmentary spots.

21. The method of claim 17, wherein the composition is administered to stimulate the synthesis of collagen and/or elastin in the skin.

22. The method of claim 17, wherein the composition is administered to increase energy metabolism and/or the production of energy by skin cells.

23. The method of claim 17, wherein the resveratrol or a derivative thereof is selected from
cis-resveratrol, and
trans-resveratrol and resveratrol esters, monomers and oligomers,
said monomers comprising at least one ester group of formula —O—CO-A, and
said oligomers being formed from monomer units linked by carbon-carbon or ether bonds, and/or of monomers cross-linked with —O—CO—R—CO—O— groups where -A represents a linear or branched, saturated or unsaturated alkyl radical with at least two carbon atoms, an aryl, aralkyl or aralkylene radical, and —R represents a saturated or unsaturated alkylene radical having 0 to 10 carbon atoms and/or 1 arylene radical having 1 to 3 rings and/or a heterocyclic radical and the diastereoisomers of these units.

24. The method of claim 17, wherein the composition comprises a conjugate of betaine with resveratrol or a derivative thereof.

25. A method of cosmetically treating or reducing signs of ageing of skin comprising applying, on the cutaneous coating or its annexes, a composition containing an association of (i) resveratrol or one or more derivatives thereof and (ii) betaine or a derivative thereof, in a cosmetically acceptable medium.

26. The method of claim 25, wherein the composition is applied to treat or reduce at least one of loss of skin firmness and/or elasticity, thinning of the skin, wrinkles and fine lines, dull complexion, skin hyperpigmentation or pigmentation heterogeneity and pigmentary spots.

27. The method of claim 25, wherein the resveratrol or one or more derivatives thereof is present in the composition in the form of a plant extract containing it.

28. The method of claim 25, wherein the betaine is a physiologically acceptable salt of betaine.

29. The method of claim 25, wherein the ratio between resveratrol and betaine is between 5:1 and 1:1000.

30. The method of claim 25, wherein the composition contains (i) from 0.001 to 10% of resveratrol and (ii) from 0.01 to 10% of betaine, said percentages being expressed in weight relative to the total weight of the composition.

31. A composition for treating ageing of skin, comprising a synergic association on the production of collagen by the cutaneous cells of
0.002 to 3% resveratrol or a derivative thereof, and
0.1 to 10% betaine
in a physiologically acceptable medium, said percentages being expressed in weight relative to the total weight of the composition.

32. The composition of claim 31, wherein the physiologically acceptable medium is a cosmetically acceptable medium and wherein the composition further contains at least one active ingredient selected from antioxidant agents, hydrating agents, exfoliating agents, agents improving skin barrier function, depigmenting agents and agents promoting cutaneous microcirculation.


SEQUENCE LISTING (4 sequences)

SEQ ID NO: 1 - DNA, 21 bp, Artificial Sequence
  primer sense Col1A1
gagggccaagacgaagacatc

SEQ ID NO: 2 - DNA, 21 bp, Artificial Sequence
  primer reverse Col1A1
cagatcacgtcatcgcacaac

SEQ ID NO: 3 - DNA, 22 bp, Artificial Sequence
  primer sens RNA18S
gagactctggcatgctaactag

SEQ ID NO: 4 - DNA, 21 bp, Artificial Sequence
  primer reverse RNA18S
ggacatctaagggcatcacag